(19) United States
(12) Patent Application Publication
(10) Pub. No.: US 2007/0093708 A1
(43) Pub. Date: Apr. 26, 2007

(54) Ultra-high-specificity device and methods for the screening of in-vivo tumors

(76) Inventors: David A. Benaron, Portola Valley, CA (US); Ilian H. Parachikov, Belmont, CA (US); Michael R. Fierro, Los Gatos, CA (US); Shai Friedland, Palo Alto, CA (US)

Correspondence Address: DORSEY & WHITNEY LLP, 555 CALIFORNIA STREET, SUITE 1000, SUITE 1000, SAN FRANCISCO, CA, 94104, US

(21) Appl. No.: 11/255,675

(22) Filed: Oct. 20, 2005

(51) Int. Cl. A61B 5/05; A61B 6/00

(52) U.S. Cl. 600/407; 600/473; 600/476

(57) ABSTRACT

A device and a method for the screening of in-vivo tumors in a target tissue are provided. The device and method provide a local measure of a risk of tumor presence in the target tissue with high specificity. The local measure may be based upon a non-linear combination of local hemoglobin and tissue oxygen saturation and other tissue characteristics.

### FIELD OF THE INVENTION

The present invention relates generally to a device and methods for performing in-vivo tumor screening. In particular, the device and methods of the present invention provide ultra-high-specificity in-vivo tumor screening using a portable, non-invasive hybrid electromagnetic and ultrasound scanner with a ultra-high-specificity logic unit that screens for the presence of a tumor with a low false-positive error rate, thus permitting a wide use in large population screening while minimizing false referrals for invasive follow-up testing.

### BACKGROUND INFORMATION

Most tumors are detected very late, typically only after a cancer is well-established. For example, the average breast tumor size in the U.S. at first discovery is 2.0 cm wide for women and 2.5 cm for men, a surprisingly large lump. In contrast, early tumors often go undetected. The continued existence of frequent late cancer diagnosis is critical, as early diagnosis resulting from widespread use of screening tests is believed to be responsible for the drop in the U.S. death rates from breast cancer and prostate cancer (the leading gender-related lethal tumors) between the 1950's to date.

The evidence suggests that early screening of tumors is critical for their early diagnosis and chances of survival for the patients screened. For example, when breast cancer patients, matched for all factors (age, risk factors, etc.), are asked at the time of their first diagnosis with cancer “Did you regularly practice screening breast self-examination?”, those that say “yes” had an average tumor size of 2.5 cm, while those that said “no” had an average tumor size of 3.2 cm. This alone would be an interesting fact, however it is made more important by the fact that these two groups of patients had very different outcomes. In the fifteen years after diagnosis, 75% of those patients who screened themselves by early self-examination survived, whereas only 57% of those who did not perform early self-screening survived.

Early screening of tumors therefore leads to smaller tumors at diagnosis and improvements in survival rates. However, their use is not as widespread as desired as their performance suffers from poor specificity. Specificity is medically-defined as “the likelihood that a normal patient will have a normal result in the absence of a tumor.” The worse the specificity, the higher the likelihood that a normal patient will have a false-positive test and be referred for additional (and unneeded) tests, such as an invasive biopsy. A false-positive test is one in which the patient tests positive for a tumor even though the patient does not, in fact, have a tumor.

Typically, tumor screening tests have been designed so as to achieve a high sensitivity to cancer—that is, to find as many cases of cancer as possible, i.e., to maximize “the likelihood that a patient with an abnormal condition, e.g., a tumor, will have an abnormal test result.” A required trade-off, based on fundamentals of sensitivity/specificity statistics, commonly referred to in the art as Receiver-Operator Curves (“ROC”), is that any increase in test sensitivity results in lower test specificity or in a higher false-positive rate.

False-positive tests have significant negative consequences, including unnecessary invasive tests, patient and family anxiety and pain, disruption of work, rising medical costs, and a fundamental loss of confidence in the medical testing itself when the workup reveals there wasn't any cancer there in the first place. In fact, in the U.S., more than ten breast biopsies are done for every breast cancer that is actually found. That means that for the vast majority of women, a positive screening test led to an unnecessary work-up. Of central importance, multiple surveys have shown that many women who had false-positive referrals to biopsy were dissatisfied with the experience. These women who then later find more lumps, their doctors who have referred patients to find only benign lumps, and the radiologists who have been burned by falsely seeing too many early lesions, are each more hesitant to declare a lesion cancerous in the future, with obvious results. This, the traditional goal of maximum sensitivity comes, in our view, at major personal and societal costs in terms of poor specificity and high false-positive rates.

To put the magnitude of the problem in perspective, consider the numbers (shown in Table 1 below) behind the current hair-trigger high-sensitivity screening tests and their attendant false-positives. The primary screening tests in widespread use for breast cancer detection are: (a) clinical breast examination done by a health specialist at a yearly check-up; (b) x-ray mammography done by a radiologist or technician yearly after 40 years of age; and (c) breast self-examination recommended for every woman monthly after age 18. Together, these three screening tests first identify the vast majority of the 225,000 new cases of breast cancer discovered each year in the U.S., while sending over 5 million women through additional workup each year. The emphasis on sensitivity to breast cancer leads a large number of biopsies and follow-up visits, as shown in Table 1 below:

TABLE 1
False-Positive Rates for Current Breast Cancer Screening Tests

| | | Best-Case |
|---|---|---|
| Breast Cancer | False-Positive | False-Positives |
| Screening Test | Rate (%) | (cases/yr) * |
| a. Clinical Examination | 4-12% ¹ | 4,000,000 |
| b. X-Ray Mammogram | 3-30% ¹ | 3,000,000 |
| c. Home Self Examination | 1-12% | 1,000,000 |

* If 100 million U.S. women use only that one test (a-c) each year
¹ National Cancer Institute, 2005, Breast Cancer (PDQ) Screening, at “www.nci.nih.gov/cancertopics/pdq/screening/breast/HealthProfessional/page3”.

Fortunately, cancerous tissues have characteristic features that differ on average, though with some overlap, with normal tissues. In Cerussi A E, Berger A J, Bevilacqua F, Shah N, Jakubowski D, Butler J, Holcombe R F, and Tromberg B J, “Sources of absorption and scattering contrast for near-infrared optical mammography,” Acad Radiol 2001;8(3):211-218, it is shown that cancerous tissues have differing average lipid, blood oxygenation, blood content, and water content from other tissues. It has also been shown that tumors are often hypoxic and/or hyperemic. However, such published methods do not constitute clinically approved (e.g., FDA- or CE-approved), enabling instruments.

For example, United States Patent Publication No. 2005/0197583 discloses the use of optics to create two optical data sets, with a processor arranged to calculate congruence of the two optical data sets to detect abnormal tissue (such as tumors in an examined tissue), but does not teach or suggest maximization of specificity as a method to perform large-scale screening with an acceptably low false-positive rate. Similarly, United States Patent Publication No. 2005/0194537, United States Patent Publication No. 2005/0020923, International Publication No. WO 1998/51209, and European Patent No. EP 1008326, all teach optical methods for monitoring cancerous tissues, but do not teach maximization of specificity, nor are adaptations of the device needed for inducing acceptance as a screening tool taught or suggested. International Publication No. WO 2005/070470 mentions the concept of sensitive and specific monitoring, but only to the extent of exploring the predictive value of the tests. It is neither suggested nor taught that a test with reduced sensitivity and increased specificity has any merit as a screening tool.

All of the above known devices are limited in being designed to have a high sensitivity. Because of the trade-off between sensitivity and specificity mentioned herein above, none of these prior art references disclose a means or arrangement designed to achieve high specificity, nor do they allow for a specific tumor detection in the setting of a large-population screening tool. In short, the prior art lacks a unit arranged and optimized for the processing of different optical information for the purpose of achieving high specificity.

Thus, there is a need to provide a device and methods for the large-scale screening of in-vivo tumors in a target tissue based on the processing of optical information from the target tissue by sacrificing sensitivity in favor of high specificity to result in an acceptably-low false-positive rate that patients and doctors could trust, that would instill confidence in the device from all users, allow the device to serve as an adjunct to current screening programs, and be widely adopted.

### SUMMARY OF THE INVENTION

In view of the foregoing, one of the objects of the present invention is to provide device and methods for the ultra-high-specificity (“UHS”) screening of in-vivo tumors.

It is another object of the present invention to provide a direct, quantitative measure or index of the presence, absence, and location of a tumor.

These and other objects of the present invention can be accomplished using an exemplary embodiment of the device and methods of the present invention in which an electromagnetic (“EM”) source, with or without an ultrasound emission capacity (“US”), produces continuous EM radiation, which is then transmitted to a target tissue site. EM radiation scattered, transmitted, fluoresced, or reemitted by the target tissue site can then be collected by an EM sensor, allowing for an index to be determined, and subsequently processed by a UHS-weighted logic unit in order to produce a measure of the presence or absence of a tumor in the target tissue site.

The device of the present invention may be coupled to a computer, to the Internet, to an intranet, or may be freestanding. As understood by one of ordinary skill in the art, devices designed to use a hybrid of US and EM radiation also fall within the spirit of the present invention.

Used as an adjunct to conventional in-vivo tumor screening tools, the device and methods of the present invention enable an earlier detection of cancer in many patients, without substantially increasing the burden of false-positive referrals. In the setting of an established screening program, traditional sensitivity-weighted detection can be sacrificed in order to add specificity-weighted detection to a screening tool without reducing the overall sensitivity of the program, with the new screening tool added as an adjunct to the established screening programs. By losing sensitivity as a guiding feature, changes can be made to the device of the present invention, such as lower spatial resolution, that facilitate manufacture, cost-effectiveness, ease of use, speed of use, and other beneficial changes.

The device and methods of the present invention as described herein below have one or more advantages.

One advantage is that a patient, physician, or surgeon can obtain real-time feedback regarding the discovery of local tumors in high-risk patients and respond early.

Another advantage is that the device and methods of the present invention may be safely deployed to patients at home or hospitals as a screening tool, to give long-term tumor-specific feedback as needed.

A further advantage is that the device and methods of the present invention can be actively coupled to a therapeutic device, such as a tumor ablation device, to provide feedback to the removal or ablation function, based upon the detection and degree of the local tumor.

Yet another advantage is that the device of the present invention may be constructed to detect tumors using EM radiation, which allows for the simple, safe, and non-electrical transmission of measuring photons.

### BRIEF DESCRIPTION OF THE DRAWINGS

The breadth of uses and advantages of the present invention are best understood by example, and by a detailed explanation of the workings of a constructed device, now in operation and tested in animals. These and other advantages of the present invention will become apparent upon consideration of the following detailed description, taken in conjunction with the accompanying drawings, in which like reference characters refer to like parts throughout, and in which:

FIG. 1A shows an exemplary schematic diagram of a hybrid EM/US in-vivo tumor screening device having a UHS logic unit in accordance with the present invention;

FIG. 1B shows an illustration of an exemplary positioning of the device shown in FIG. 1A in relation to a human subject undergoing an in-vivo tumor screening in accordance with the present invention;

FIGS. 2A-E show exemplary schematic diagrams of various configurations of the sensor shown in FIG. 1A and constructed in accordance with the present invention;

FIGS. 3A-E show exemplary illustrations of how an in-vivo tumor screening can be conducted on a patient using EM but no US radiation;

FIG. 4 shows an exemplary illustration of how an in-vivo tumor screening can be made more accurate at one location by depth using depth-focus-scanned US modulation;

FIG. 5 shows a flow diagram of an exemplary embodiment of a in-vivo tumor screening performed in accordance with the present invention; and

FIG. 6 shows an exemplary data set collected on human tumors using an exemplary device constructed in accordance with the present invention.

### DETAILED DESCRIPTION OF EMBODIMENTS OF THE INVENTION

Generally, in accordance with exemplary embodiments of the present invention, a device and methods for the ultra-high-specificity (“UHS”) screening of in-vivo tumors in a target tissue site are provided. Tumors, as used herein, generally refer to a malignant tissue, or other type of cancer, to be diagnosed in a tissue, which may be any material from a living animal, plant, viral, or bacterial subject, with an emphasis on mammals, especially humans. A target tissue may be a tissue material to be detected, imaged, or studied. In the accompanying examples described herein below, one target tissue may be the breast.

In accordance with the present invention, the screening of in-vivo tumors can be applied in a large population. The device of the present invention can be used as an adjunct screening tool that is used in addition to other standard screening tools already in use. An adjunct screening tool has the disadvantage that it adds to the number of tests performed, but it also has the compelling advantage that, if used, the percent of patients with a tumor who are indeed detected early can only increase.

As described in further detail herein below, the device of the present invention sacrifices sensitivity in favor of specificity. Sensitivity, as used herein, generally refers to the a priori probability that a patient with an abnormal condition, such as a tumor, will have an abnormal screening test result. That is, sensitivity refers to the probability that a patient having a disease will be correctly screened as having the disease. Sensitivity may be expressed as one minus the false-negative rate, i.e., the rate of screening tests resulting in a negative diagnosis of a disease when the patient, in fact, has the disease.

Specificity, as used herein, generally refers to the a priori probability that a normal patient will have a normal test result. Specificity may be expressed as one minus the false-positive rate, i.e., the rate of screening tests resulting in a positive diagnosis of a disease when the patient, in fact, does not have the disease.

The device of the present invention can generally be referred to as a ultra-high-specificity (“UHS”) device. As used herein, a UHS device attempts to give a strong degree of certainty to a positive diagnosis at the cost of missing some patients who have the disease. A UHS device can minimize its false-positive rate and achieve a true-positive rate, i.e., the rate of screening tests resulting in a positive diagnosis of a disease when the patient, in fact, has the disease, of at least one-third in a given large screening population.

A key aspect of a UHS device is that it does not aim for maximum detection, but rather, for maximizing the chances that a positive screening test will be accurate. In contrast, most, if not all, in-vivo tumor screening tests strive for maximum detection of tumors, i.e., maximum sensitivity. For example, it is conventionally acceptable in medicine that there are five to ten breast biopsies performed for every breast cancer found. This means that many patients without breast cancer have been identified as having breast cancer by the conventional breast cancer screening tests available today. A UHS device may miss some or even many of the patients with breast cancer, but it has the unique and powerful advantage that when a UHS test is positive for cancer screening, the probability that the patient has breast cancer (or whatever cancer is being screened for) will be large, likely at least one-third or one-half, or even higher if desired.

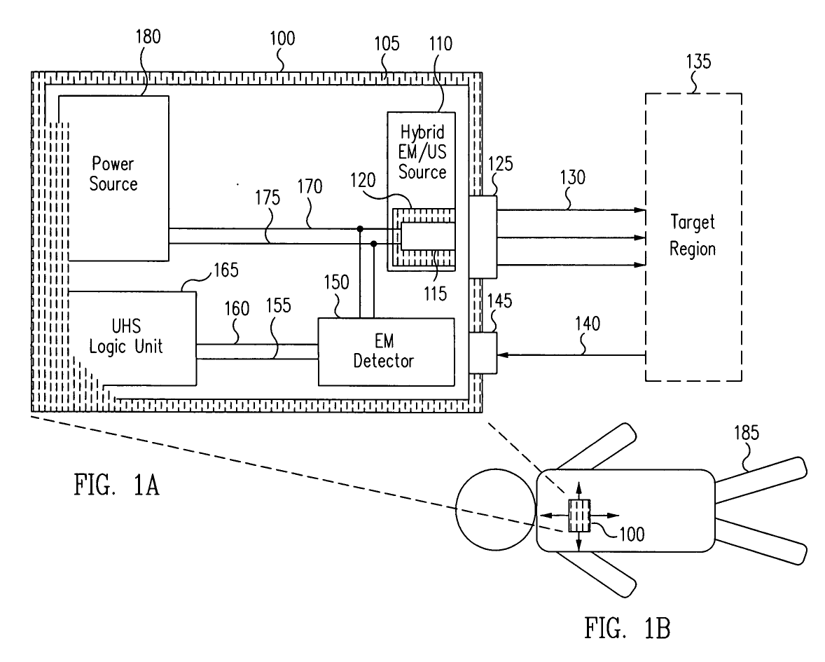

Referring to FIG. 1A, an exemplary schematic diagram of a hybrid EM/US in-vivo tumor screening device having a UHS logic unit in accordance with the present invention is provided. In-vivo tumor screening device 100 is shown surrounded by soft silicone exterior 105. Typically, exterior 105 is constructed from approved Class VI materials as recognized by the United States Food and Drug Administration or other medical device regulatory agencies, such as polyethylene or surgical steel. Portions of device 100 may protrude as needed from this shell within the spirit of the invention, provided that the protruding parts themselves are biocompatible.

Within device 100, hybrid US/EM source 110 is illustrated in its component parts. Broad spectrum infrared radiation is produced and emitted by a red LED coated with a broadband infrared phosphor to form broadband source 115. Broadband source 115 is embedded into a plastic beam-shaping mount using optical clear epoxy 120 to allow EM radiation generated in source 115 to pass forward as shown by radiation path vectors 130, with at least a portion of this radiation optically coupled to target region 135, thus illuminating target region 135. Target region 135 may be a living tissue site desired to be screened for tumors by device 100, such as breast tissue or any other potentially cancerous tissue. EM source 110 also may have two electrical connections such as electrical connections 170 and 175, connecting EM source 110 to power source 180. In one exemplary embodiment, power source 180 may be a battery.

As used herein, optically coupling the EM radiation generated by broadband source 115 to target region 135 generally refers to the arrangement of two elements, e.g., broadband source 115 and target region 135, respectively, such that EM radiation exiting the first element interacts, at least in part, with the second element. This may be in the form of free-space (unaided) transmission through air or space, or may require use of intervening optical elements such as lenses, filters, fused fiber expanders, collimators, concentrators, collectors, optical fibers, prisms, mirrors, or mirrored surfaces.

A portion of the radiation reaching target region 135 interacts with, i.e., is spectroscopically, fluorescently, rotationally, temporally, or otherwise affected by, the tissue in target region 135 and returns to device 100, as shown by radiation path vector 140, via optical collection window 145. Collection window 145 in this embodiment may be a glass, plastic, or quartz window, but can alternatively be merely an aperture, or even a lens, as required. EM radiation returning from target region 135 via optical collection window 145 then strikes EM detector or sensor 150, where it is sensed and detected.

Further, ultrasound emission can be added to source 110, such as for scanning through the tissue, in order to add an oscillatory AC signal to the overall EM radiation, thereby labeling the light that travels through a specific volume of tissue on which the ultrasound is focused. Such focusing of ultrasound to produce a spatial tagging is known in the art (e.g., DiMarzio 2003, EP 1 008 326)

Sensor 150 may consist of a number of discrete detectors configured to be wavelength-sensitive, or may be a continuous CCD spectrometer, with entry of EM radiation by wavelength-controlled gratings, filters, or wavelength-specific optical fibers. In any event, sensor 150 transmits a tumor-specific signal related to the detected EM radiation backscattered from target region 135, producing an electrical signal (in this case, a digital signal) sent via wires 160 and 165 to UHS logic unit 165 for generation of a target signal.

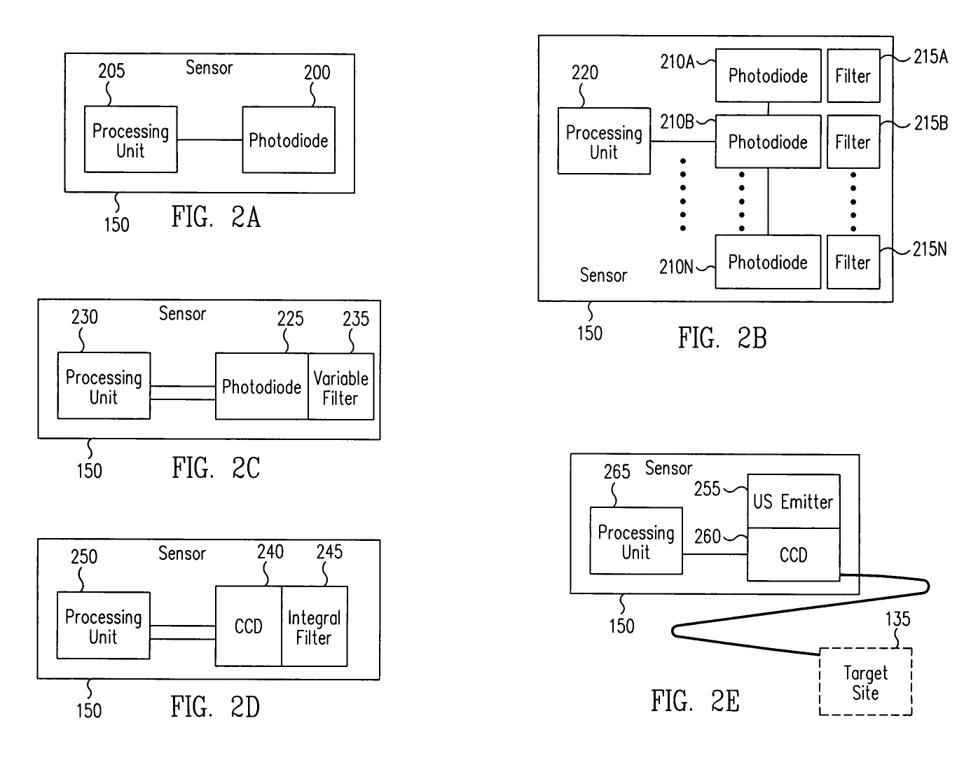

Referring now to FIGS. 2A-E, exemplary schematic diagrams of various configurations of the sensor shown in FIG. 1A and constructed in accordance with the present invention are provided. In one configuration, shown in FIG. 2A, sensor 150 is merely single photodiode 200 and processing electronics unit 205. Photodiode 200 is made wavelength sensitive through the design of LED 115 as a cluster of LEDs of different wavelengths, each emitting at a different time or modulation frequency to allow decoding of the illuminating wavelength by photodiode 200 and processing electronics unit 205. Alternatively, as shown in FIG. 2B, sensor 150 may comprise a set of different photodiodes 210a-n, each with filters 215a-n, allowing each photodiode to be sensitive to only one wavelength range, again allowing decoding of the sensed light by wavelength by processing electronics unit 220.

Alternatively again, as shown in FIG. 2C, sensor 150 may be single photodiode 225 with electronically variable filter 230, allowing the wavelength transmitted to be selected and processed by processing electronics unit 235. An example of such a tunable filter is the VariSpec™ device sold by Cambridge Research, Inc., of Cambridge, Mass. In yet another configuration, as shown in FIG. 2D, sensor 150 may be CCD chip 240 with integrated filter window 245 that varies over its length, allowing only certain wavelengths to reach each portion of CCD 240, allowing decoding of the illuminating wavelength by processing electronics unit 250.

Another configuration may be the hybrid electromagnetic and acoustic embodiment shown in FIG. 2E. In FIG. 2E, sensor 150 comprises CCD chip 255 and depth-focused ultrasound emitter 260 attached to CCD 255 in a linear array to modulate tissue at varying depth with an ultrasonic wave to allow for a depth-resolved target signal to be constructed.

It should be understood by one of ordinary skill in the art that the sensor configurations illustrated in FIGS. 2A-E are provided for purposes of illustrations only, in order to demonstrate the flexibility of sensors constructed in accordance with the invention. The sensor configurations illustrated in FIGS. 2A-E are not intended to be all-encompassing nor restrictively limiting by omission. Additional sensor configurations may be used without deviating from the principles and embodiments of the present invention.

The target signal generated by sensor 150 may be enhanced through use of known optical techniques, including the use of a contrast agent, scattering, absorbance, phosphorescence, fluorescence, Raman effects, or other known spectroscopy techniques, provided only that such techniques can be applied in a manner to perform UHS in-vivo tumor sensing, detection, localization, or imaging. The target signal could be a function of, for example, capillary saturation and blood content, both known to change from normal tissues during tumor initiation and growth.

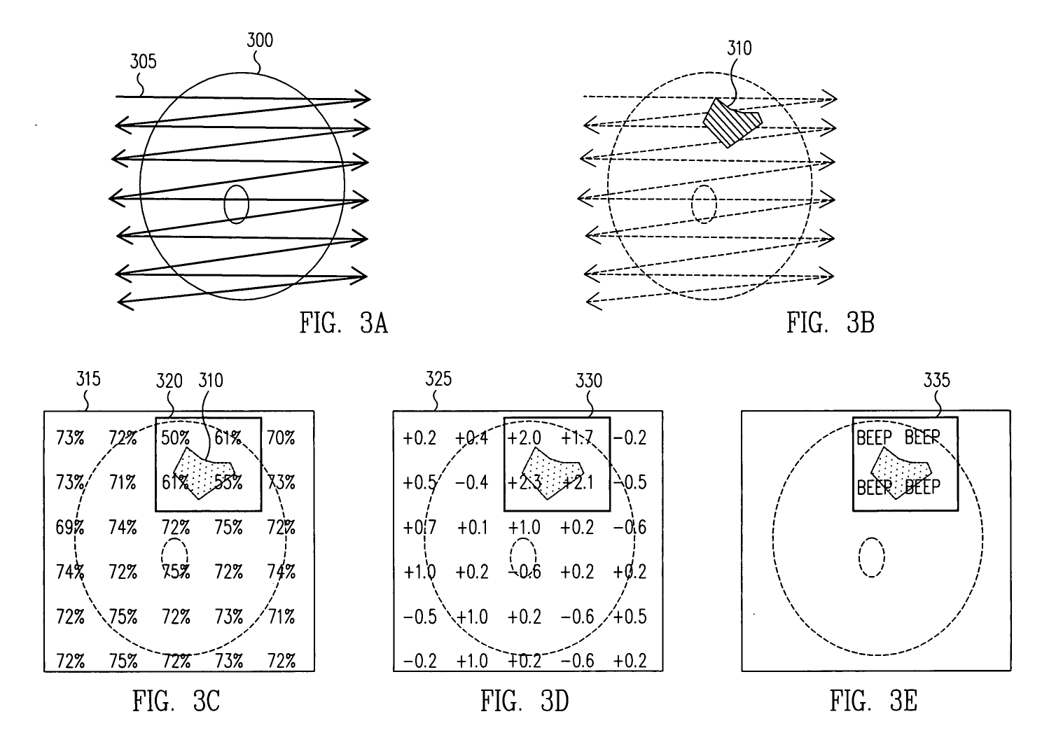

Referring now to FIG. 1B, an illustration of an exemplary positioning of the device shown in FIG. 1A in relation to a human subject undergoing an in-vivo tumor screening in accordance with the present invention is provided. Device 100 is shown as placed on the chest of a breast cancer screening subject 185. In this exemplary embodiment, device 100 is shown to be sufficiently small and light so as to be placed over a small portion of the chest of subject 185, and to perform the scans illustrated in FIGS. 2A-E and FIG. 3, as described in more detail herein below.

In this exemplary embodiment, it is desired to test a target tissue for the presence of a tumor. When device 100 is turned on, EM source 110 begins to illuminate target region 135, in this case the breast tissue of subject 185. Subject 185 may be a living subject, and as such, is provided for illustrative purposes in understanding the operation and use of the present invention.

Once device 100 is placed on subject 185, sensor 150, which can be an embedded spectrophotometer, receives backscattered EM radiation and separates and measures the incoming EM radiation by wavelength, that is, sensor 150 analyzes the incoming EM radiation to determine how much light is reflected for each wavelength transmitted by broadband source 115. Analysis of this incoming radiation is then sent to the Ultra-High-Specificity (“UHS”) logic unit 165 for generation of a direct, quantitative measure or index of the presence, absence, and location of a tumor, as described in more detail herein below.

To generate an index indicating the presence, absence, and location of a tumor, device 100 may scan target tissue region 135 several times. Various scanning patterns may be used, with each one collecting numerous data points of EM radiation reflected by target tissue 135 upon receiving broadband light from broadband source 115. Sensor 150 determines for each wavelength transmitted, the amount of light that was reflected back. For example, device 100 may be used as a screening device to detect breast tumors by scanning breast tissue several times to cover the breast area.

Referring now to FIGS. 3A-E, exemplary illustrations of how an in-vivo tumor screening can be conducted on a patient using EM but no US radiation is provided. One scanning pattern using device 100 across breast 300 of subject 185 is illustrated in FIG. 3A. Scanning pattern 305 is merely illustrative of one of the many possible patterns, but by no means is intended or implied to be the best or only scanning pattern.

In FIG. 3A, breast 300 is screened using a back-and-forth scanning pattern over the brief period of time required to move device 100 in pattern 305 across breast 300, with sufficient dwelling time as required to collect and process the optical data. This back-and-forth pattern is intended to give a full surface scan of, and maximal volumetric coverage to the tissue within, breast 300. As noted above, other patterns equivalent or superior to pattern 305 could be used, and such other patterns could reasonably include the sides or folds between the breast and the chest, or other patterns as deemed useful and relevant, such that a high specificity of detection is achieved.

In FIG. 3B, breast 300 and back-and-forth scanning pattern 305 of FIG. 3A are shown as dotted lines to allow tumor 310 to be clearly seen. Note that tumor 310 of FIG. 3B is a large tumor, partially crossed in more than one back-and-forth sections. This will not necessarily be the case with smaller tumors, but will suffice for this example. As device 100 is passed over breast 300 in scanning pattern 305, UHS logic unit 165 processes the optical information to determine an indication of the presence, absence, and location of a tumor for different regions scanned by device 100 with scanning pattern 305.

For example, in FIG. 3C, a grid of tissue saturations determined by UHS logic unit 165 is shown for different regions of scanning pattern 305 as saturation grid 315. Note that the grid values are shown as if they are determined exactly on a rectilinear two-dimensional saturation grid. Grid 315 may not exist as a precise two-dimensional grid in practice (though it can be created, if desired), as such values are determined continuously and in real time during scanning, wherever the scan is taken. However, for this example, grid 315 serves as a good illustration of the types of processing possible, without deviating from the principles and embodiments of the present invention.

As shown in grid 315, tissue saturation values can be seen to be lower than elsewhere in low saturation region 320, namely the four grid squares shown over tumor 310. A saturation threshold can be used by UHS logic unit 165 to produce a beeping when device 100 is over region 310 as illustrated in FIG. 3E as alert region 335. The precise saturation values used to determine the alert (by beeping) by logic unit 165 may consist of a function of quantitative saturation, relative saturation compared to tissue at the same site at a different depth, tissue saturation at a different site on the scanning grid, tissue at a same breast location but on the opposite breast, or any other calculated, pre-specified or actively and adaptively-determined, saturation value.

In FIG. 3D, a different UHS threshold value is shown. In this figure, relative tissue hemoglobin concentration is plotted on the same two-dimensional grid over the same regions of scanning pattern 305 as was used for saturation grid 315. This new grid is shown as hemoglobin grid 325. Note again that the values are plotted as if determined on a precise two-dimensional grid for illustrative purposes only. In this figure, relative tissue hemoglobin values can be seen to be lower in high hemoglobin region 330, namely the same four grid squares over tumor 310. A saturation threshold can be used by logic unit 165 to produce a beeping when device 100 is over region 310. As with saturation, hemoglobin values used in the determination of beeping by UHS logic unit 165 may be quantitative hemoglobin concentration, or relative concentration as compared to tissue at the same site at a different depth, tissue at a different site on the scanning grid, tissue at a same breast location but on the opposite breast, or any other calculated, pre-specified or actively and adaptively-determined, hemoglobin value.

Hemoglobin saturation values distributed as an image may be obtained using multiple, imaging receivers and/or emitters, and software for solving for a diffusion-weighted image. Alternatively, it may be advantageous to include ultrasound emitters to tag the optical signal with a depth-related feature. Both of these approaches are well-known in the art.

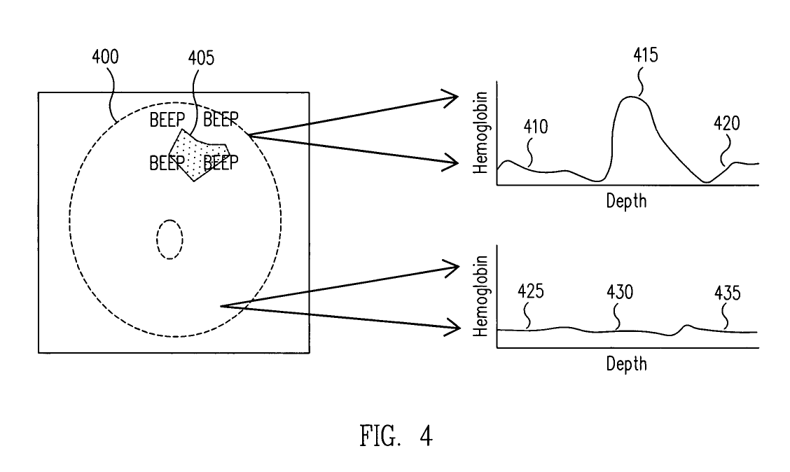

Referring now to FIG. 4, an exemplary illustration of how an in-vivo tumor screening can be made more accurate at one location by depth using depth-focus-scanned US modulation is provided. FIG. 4 illustrates one example of data derived from the use of ultrasound to optically label the acquired data with a depth window, allowing the signal to be swept from shallow to deep at the same surface location of the tissue being screened. This allows each pixel in the grid to obtain a depth component, effectively turning a two-dimensional scan into a three-dimensional scan.

As shown in FIG. 4, two grid square regions of breast 400 are scanned. The first region is partially over tumor 310. As the ultrasound signal is swept from the surface to deeper depths, the hemoglobin signal can be seen to increase from shallow depth 410 to peak at intermediate depth 415 and to decline again at deepest depth 420. In this case, the tumor is located under the surface, and the depth scan has allowed the optical test to pass from above the tumor, into the tumor, and finally below the tumor. The maximum hemoglobin concentration at intermediate depth 420 is passed to UHS logic unit 165 to cause device 100 to beep, indicating the presence of tumor at some depth in that grid square. A depth signal, e.g., in millimeters, could reasonably be displayed to the user, for assistance in tracking the tumor.

Referring now to the second and lower grid region scanned in FIG. 4, the hemoglobin signal can be seen to remain stable from shallow depth 425 to intermediate depth 430 to deepest depth 435. In this case, there is no tumor found on the depth scan. The maximum hemoglobin concentration in this grid square is passed to UHS logic unit 165, and no alarm is generated.

One additional option is the use of an intravenous or subcutaneous injection of an optical contrast agent, such as indocyanine green. As tumors have immature and leaky blood vessels, an indocyanine injection would clear rapidly from the bloodstream, but remain in the tissue where it has extravasated, such as in a tumor. Then, a depth-resolved scan for indocyanine green rather than for hemoglobin (absorbance or fluorescence based) would produce data identical in form to that seen in FIG. 4, with a depth-related peak in signal over a tumor and a more flat and bland scan over normal tissue.

There are many imaging-generation and depth-focusing methods known in the art, some demonstrated here for illustration, without an intent of limitation by omission, and these approaches can be used by those skilled in the art to provide further specificity or functionality to the scan, including, without limitation, spatially-resolved scanning, time-resolved scanning, and frequency-domain scanning.

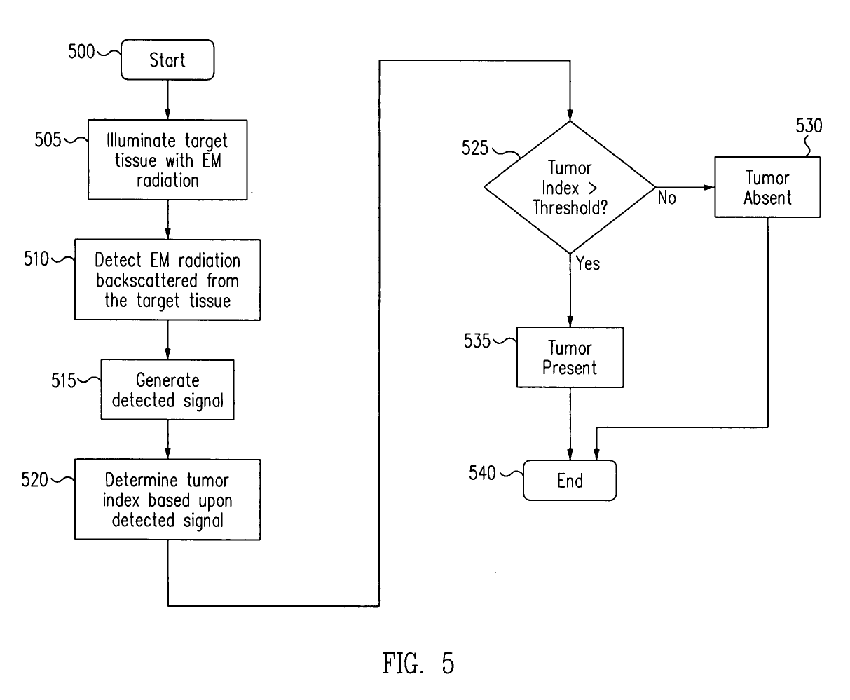

Regardless of the approach and index measure used to detect the presence of a tumor, the purpose of UHS logic unit 165 is to provide a highly-specific determination of the presence of the tumor. Because device 100 is a screening device, the presence of even a moderate rate of false-positives will result in a large increase in the referral rate, as will be described below with reference to FIG. 5. An increase in specificity of a tumor screening test (i.e., a reduction in false-positives) can almost always be achieved by a lessening of the detection rate (i.e., the sensitivity), as related mathematically by the Receiver-Operator Curve. That is, a UHS test can be designed to miss more positive tests than typically required by the test.

Because of this purpose, to improve specificity even at the cost of sensitivity, an ultra-high-specificity test is counter-intuitive, as it is obvious to those in the field that one would rarely, if ever, intentionally reduce the sensitivity of a tumor screening test. In view of this, UHS logic unit 165 uses known as well as novel measures to produce an inventive method to allow for in-vivo tumor screening with a very high specificity.

UHS logic unit 165 index of the presence of a tumor could be as simple as “if the hemoglobin content of the target tissue is more than 5 standard deviations above normal tissue nearby, then the target tissue screening test is positive for cancer at that target site.” In other cases, a non-linear combination of several features may be used, such as “if (a) the hemoglobin content of the target tissue is more than 5 standard deviations above normal tissue nearby, OR (b) the tissue hemoglobin saturation is more than 5 standard deviations below normal tissue nearby, OR (c) the sum of the two above-listed standard deviations is above 5, then the target tissue screening test is positive for cancer at that target site.”

This non-linear combination of features used by UHS logic unit 165 can be used to produce a “tumor index,” i.e., a numeric indication of whether the patient has a tumor or not. Such a tumor index is at least one step removed from direct physiologic measures such as total hemoglobin or tissue saturation. In this case, for example, one index could reasonably be a sum of standard deviations of the hemoglobin and saturation values. An index sum of 6 or more might then be used to indicate the presence of tumor, but the value of “6” is only an indirect function of tumor hemoglobin and tumor tissue saturation, rather than a direct representation of these values themselves. One advantage of such an index is that published peer-reviewed studies could then be used to fine-tune or adjust the UHS specificity threshold in different populations of patients at risk for cancer, such as women with a history of cancer may use the threshold of 6, while those without a history of cancer may require a threshold index of 8 in order to consider the screening test positive.

Features used to generate a tumor index could reasonably include any tumor-related measurable feature, including but not limited to hemoglobin content, tissue saturation, fat content, water content, the presence of leaky new capillary vessels, tissue necrosis, temperature, increased DNA content, increased cell size, nuclear scattering, or any measurable parameter of tumors, but especially those features quantifiable using optical and/or ultrasonic means.

It is important that UHS logic unit 165 does not need to have a fixed threshold for tumor/normal screening classification. For example, a small tumor may produce only a small change in the tested values as device 100 is moved across a breast under testing. For this reason, there may be an adjustable tumor threshold setting that results in an initial positive test. Then, over each region where device 100 has beeped, a UHS test threshold can be used to more carefully test those regions of higher suspicion.

Referring now to FIG. 5, a flow diagram of an exemplary embodiment of a in-vivo tumor screening performed in accordance with the present invention is provided. In-vivo tumor screening starts in step 505 with device 100 illuminating a target tissue with EM radiation. The user, e.g., a medical examiner or a patient, scans device 100 across the target tissue in a scanning pattern such as scanning pattern 305 shown in FIG. 3A. At each location scanned, device 100 detects EM radiation backscattered from the target tissue in step 510 and generates a detected signal based on the backscattered EM radiation in step 515.

The detected signal is sent to UHS logic unit 165 for processing. As described herein below, UHS logic unit 165 may use tissue hemoglobin content and saturation, tissue myoglobin content and saturation, other tissue characteristics, and a combination thereof to determine a tumor index in step 520 based upon the detected signal. In step 525, the tumor index is compared to a threshold. This threshold may be a number predetermined by the prescribing physician based upon peer-reviewed studies, or may be a fixed numeric threshold pre-programmed into device 100. If the tumor index is above the threshold in step 530, device 100 does not beep and no indication that a tumor is present is given to the user. Otherwise, device 100 beeps over the scanned location in step 535, thereby indicating to the user that a tumor is present in the scanned location with ultra-high-specificity. As used herein, ultra-high-specificity is achieved when at least one-third (if not the majority or even two-thirds) of positive screening tests are true positives.

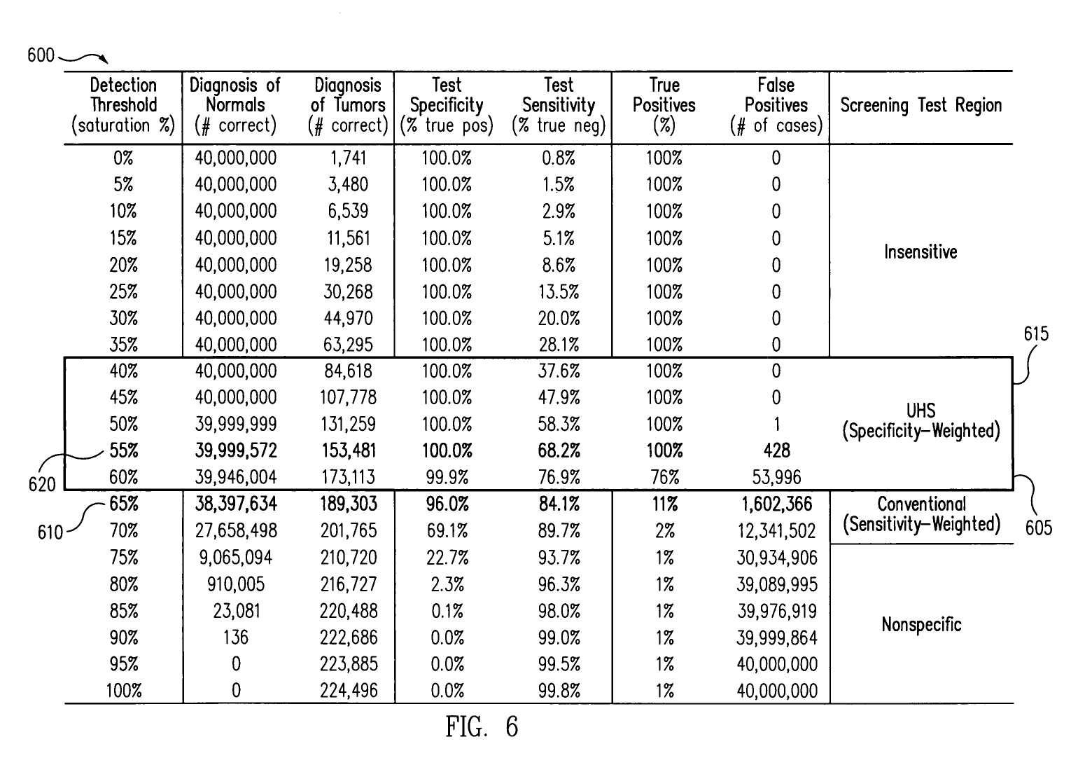

The interplay between sensitivity and specificity that drives the selection of a threshold may be best understood by an example. Referring to FIG. 6, an exemplary data set collected on human tumors using an exemplary device constructed in accordance with the present invention. In this example, tissue oxygenation levels from gastrointestinal polyps are used to predict the effect of a similar tissue oxygen level being used to predict the presence of breast cancer in a breast cancer scan with very high specificity. In data collected by on living patients, the oxygenation of normal versus cancerous gastrointestinal polyps during endoscopy using visible EM radiation optical spectroscopy was measured at 72±4% for normal tissue, and 46±19% for tumors. These values may be used to guide the selection of a threshold for determining the oxygen levels at which the breast tissue may be cancerous.

Using this endoscopic tumor data collected on human subjects, a tumor index, in this case related to tissue saturation, can be established. If the index falls below the threshold (or above the threshold, as applicable), then the tissue is considered “tumorous;” otherwise, the tissue is considered “normal.” The difference between conventional high sensitivity versus the present invention's high specificity threshold can be seen in Table 600 shown in FIG. 6. Based upon a sample population of 40 million subjects, with 225,000 actually having breast cancer, the sensitivity and specificity of the test using device 100 can be determined at varying threshold tumor indices, as illustrated in FIG. 6.

Conventional cancer screening approaches would suggest maximizing sensitivity, as shown by region 605. Region 605 is labeled in the last column as the “Conventional Sensitivity-Weighted” screening region. In region 605, sensitivity is reasonably maximized at a tumor saturation index threshold of about 65-70%, where 84%-94% of the tumors would be detected. An example of conventional optimized sensitivity is highlighted as bold entry line 610 in region 605. At this sensitivity-optimized threshold, there would also be over 1,600,000 false-positives, with only 11% of the positive results being true positives, and the remainder being false-positives. This is representative of the current breast screening approaches, in which the specificity is 96% and only 1 in 10 referrals are true positives.

In contrast, in accordance with the present invention, specificity may be maximized using UHS logic unit 165 by reducing the number of tumors detected in direct opposition to conventional wisdom, as shown in region 615. Region 615 is labeled in the last column as the “UHS Specificity-Weighted” screening region. An illustrative example of an optimized specificity tumor index is shown as bold entry line 620. In this specificity-optimized threshold, the saturation threshold tumor index is 55%. With this threshold, the sensitivity falls to 68%, well below that of the current screening tests, while in return the specificity rises to nearly 100%. Two-thirds of the tumors would still be discovered early, which goes against conventional art by rejecting one-third of tumors, but only 428 cases would be referred for additional testing that was not, in retrospect, required using a 55% threshold. In this scenario, the true-positives may comprise the majority of referrals.

Example 620 rejects more tumors than would be acceptable under most standard methods for optimizing cancer screening tests. For a UHS device, such as device 100 in accordance with the present invention, a good rule of thumb would be that at least one-third (if not the majority or even two-thirds) of positive screening tests are true positives in order for the device to be considered a UHS device. Such a low false-positive rate would result in a high degree of trust among physicians and patients.

Of course, with UHS, one needs to be concerned about what happens to those women with tumors that are NOT detected. First, over the next few years, those tumors not detected will continue to develop, and ultimately the tumor will evolve sufficiently so as to be detectable. This occurs as the EM signal becomes stronger as tumors grow (more blood to measure), and as tumors mature (more oxygen difference from normal tissue). Also, as tumors grow, they will become more detectable by standard screening methods (mammography, clinical exam, and self-exam). Ultimately, the tumor will be detected by one of these methods, either the new UHS method or the conventional method. On average, women as a group will continue to detect their tumors earlier when a UHS method is added to the screening regimen.

As shown in FIG. 6, only one feature (optical tissue hemoglobin saturation) was used to determine the tumor index. A tumor index of high specificity for the risk or presence of cancer could be constructed using only total hemoglobin, or both saturation and hemoglobin. Further, one or more of absorbance, scattering, blood hemoglobin content, lipid content, water, temperature, fluorescence, enhancement with optical contrast, or other optical or optical plus ultrasonic features may also be used to determine a tumor index, provided only that the determination is arranged to occur with a very high specificity sufficient to allow for widespread screening use. One example of a tumor index rule would be: “if the oxygen saturation is more than three standard deviations (“S.D.”) below normal AND/OR the blood hemoglobin content is more than three S.D. above normal, then the tissue is at risk for being cancerous.” The optical index could be the S.D. number for the saturation added to the S.D. number of the hemoglobin, for a combined tumor optical index. Then, the UHS determination would be made on a clinically-validated threshold, say a score of three or higher, to be suggestive of the risk of the presence of cancer.

Other UHS logic processing means (including, without limitation, the following: adaptive filters, weighted decision tree nodes, fuzzy logic, look-up tables, adaptive thresholds, left/right breast difference comparison, spatial changes in optical values, and the like) can be implemented in order to provide reliability and robustness to the selection of the tumor index and threshold, all within the spirit of the invention.

Further, one can use the tumor index to test for the presence of cancer in an operating room. In this case, tissue could be cut away until the device no longer detects cancer in the surgical field so that a more effective cancer resection can be achieved.

As understood by one of ordinary skill in the art, device 100 may need some form of record keeping, calibration standard, or other component that wears out. In this case, UHS device 100 may come in a kit, with reusable and disposable parts, or merely as a disposables refill kit. In a similar manner, blood glucose meters come with disposable lab test strips as a refill kit, while the glucose monitor itself is replaceable.

A UHS tumor screening and detection device for breast and other cancer deployment into a large population in a noninvasive manner has been described herein above. The device may be used on a broad array of target tissue sites, including detecting breast and gastrointestinal tumors. A working device has been built and tested, in which a broadband EM source and integrated collimating optics produce continuous EM radiation, which is then directly transmitted to a target tissue site. EM radiation scattered and/or transmitted by the target site is collected by a sensor, i.e., sensor 150, allowing for an index of cancer to be determined, and subsequently compared by a UHS logic unit, i.e., UHS logic unit 165. Power may be provided by an internal power source. The entire handheld device is encapsulated by a biocompatible shell. Used as an adjunct to conventional cancer testing, device 100 allows for an earlier detection of cancer in many patients, without substantially increasing the burden of false-positive referrals.

It should be understood by one of ordinary skill in the art that the present device may be coupled to a computer, to the internet, to an intranet, or may be freestanding. Other means to focus the beam, such as ultrasound/optical hybrid devices fall within the spirit of the ultra-high-specificity present invention.

Devices built in accordance with the present invention have not been previously described, nor successfully commercialized, and represent an important advance in the art. Such devices have immediate application to several important problems, both medical and industrial, and thus constitute an important advance in the art.

The foregoing descriptions of specific embodiments and best mode of the present invention have been presented for purposes of illustration and description only. They are not intended to be exhaustive or to limit the invention to the precise forms disclosed. Specific features of the invention are shown in some drawings and not in others, for purposes of convenience only, and any feature may be combined with other features in accordance with the invention. Steps of the described processes may be reordered or combined, and other steps may be included. The embodiments were chosen and described in order to best explain the principles of the invention and its practical application, to thereby enable others skilled in the art to best utilize the invention and various embodiments with various modifications as are suited to the particular use contemplated. Further variations of the invention will be apparent to one skilled in the art in light of this disclosure and such variations are intended to fall within the scope of the appended claims and their equivalents. The publications referenced above are incorporated herein by reference in their entireties.

What is claimed is:

1. A device for the screening of in-vivo tumors in a target tissue, the device comprising:
an electromagnetic source for illuminating the target tissue;
an electromagnetic detector arranged to detect the presence of at least one tumorous feature in the target tissue based on electromagnetic radiation backscattered from the target tissue and to generate a detected signal; and
a logic unit for generating a tumor index based upon the detected signal, the tumor index determined to detect the presence of the at least one tumorous feature with high specificity.

2. The device of claim 1, wherein the electromagnetic source comprises a broadband source.

3. The device of claim 1, wherein the electromagnetic detector comprises a detector selected from any one or more of: a photodiode; at least one photodiode coupled to at least one filter; a photodiode coupled to a variable filter; a CCD; or a CCD coupled to a depth-focused ultrasound emitter.

4. The device of claim 1, further comprising a power source.

5. The device of claim 1, wherein the tumor index comprises a function selected from any one or more of a function of at least one of tissue hemoglobin content and tissue myoglobin content; a function of at least one of tissue hemoglobin saturation and tissue myoglobin saturation; a function of at least one of tissue fat and water content; a function of at least one of compressibility, elasticity, rigidity, or stiffness; a non-linear function of tissue hemoglobin content and tissue hemoglobin saturation; or a function of a combination of tissue characteristics.

6. A method for the screening of in-vivo tumors in a target tissue, the method comprising:
illuminating the target tissue with an electromagnetic source;
detecting electromagnetic radiation backscattered from the target tissue to generate a detected signal; and
generating a tumor index based upon the detected signal, the tumor index determined to detect the presence of at least one tumorous feature in the target tissue with high specificity.

7. The method of claim 6, wherein illuminating the target tissue with an electromagnetic source comprises illuminating the target tissue with a broadband source.

8. The method of claim 6, wherein detecting electromagnetic radiation backscattered from the target tissue to generate a detected signal comprises using a detector selected from one or more of: a photodiode; at least one photodiode coupled to at least one filter; a photodiode coupled to a variable filter; a CCD; or a CCD coupled to a depth-focused ultrasound emitter.

9. The method of claim 6, wherein the tumor index comprises a function selected from one or more of: a function of at least one of tissue hemoglobin content and tissue myoglobin content; a function of at least one of tissue hemoglobin saturation and tissue myoglobin saturation; a function of at least one of tissue fat and water content; a function of at least one of compressibility, elasticity, rigidity, or stiffness; a non-linear function of tissue hemoglobin content and tissue hemoglobin saturation; or a function of a combination of tissue characteristics.

10. The method of claim 6, further comprising illuminating the target tissue with ultrasound radiation.

11. The method of claim 10, wherein generating the tumor index based upon the detected signal comprises using depth information provided with ultrasound radiation backscattered from the target tissue.

12. A method of treating a tumor in a living tissue, comprising the steps of:
(a) illuminating a target site with an electromagnetic source;
(b) detecting electromagnetic radiation backscattered from the target site to generate a detected signal;
(c) generating a tumor index based upon the detected signal, the tumor index selected so as to determine a risk of a tumor being present in the target site with high specificity; and
(d) performing an interventional therapeutic medical action based upon the risk of a tumor being present in the target site.

13. An in vivo screening apparatus comprising: an electromagnetic illuminator and detector arranged to detect the presence of at least one cancer-specific feature in a target tissue volume, and for generating a detected signal; and a high-specificity logic unit configured to have ultra-high specificity with regard to the presence or absence of a tumor in said tissue volume, and for generating an ultra-high-specificity tumor index output signal based upon the detected signal.

14. A method of in vivo cancer screening comprising the steps of: providing an electromagnetic sensor configured to be specific to the presence of at least one cancer-specific feature in a target tissue volume, illuminating and detecting electromagnetic radiation from a target tissue; and generating a tumor index output, said output signal generated by an ultra-high specificity logic unit arranged so as to allow for widespread screening use with ultra-high-specificity.

15. A method of monitoring or detecting breast cancer in a living tissue; comprising the steps of:
(a) illuminating a target site with EM and US radiation emitted from a device;
(b) detecting ultrasound-modulated EM radiation returning from the site using the device; and
(c) determining an Ultra-High-Specificity (UHS) measure that is a function of the risk of the presence of a tumor at the site based upon the detected modulated EM radiation.

16. A method of treating a tumor in a living tissue, comprising the steps of:
(a) illuminating a target site with EM radiation emitted from a device;
(b) detecting EM radiation returning from the site using the device;
(c) determining an Ultra-high-Specificity (UHS) measure that is a function of the risk of presence of a tumor at the site based upon the detected EM radiation; and
(d) performing interventional therapeutic medical action based upon said risk of presence of the tumor.

17. The method of claim 16 further comprising:
repeating steps (a) through (d) as desired.

18. A detection device for in vivo detection of a tumor in living tissue, characterized in that said detection device is configured to have high specificity for the presence or absence of a tumor in the living tissue, as opposed to high sensitivity for the presence or absence of the tumor.
